(19) United States
(12) Patent Application Publication
(10) Pub. No.: US 2017/0247359 A1
(43) Pub. Date: Aug. 31, 2017

(54) PROCESS FOR THE PREPARATION OF CANAGLIFLOZIN

(71) Applicant: MYLAN LABORATORIES LTD., Hyderabad (IN)

(72) Inventors: Siva Ram Prasad Vellanki, Hyderabad (IN); Raja Babu Balusu, Hyderabad (IN); Ramakrishna Pilli, Hyderabad (IN); Rajeswara Roa Jawaji, Hyderabad (IN); Ankama Nayudu Annadasu, Hyderabad (IN)

(21) Appl. No.: 15/508,274

(22) PCT Filed: Sep. 4, 2015
(86) PCT No.: PCT/IB2015/056751

§ 371 Date: Mar. 2, 2017

(30) Foreign Application Priority Data
Sep. 5, 2014 (IN) 4366/CHE/2014

(51) Int. Cl. C07D 409/10; C07F 7/18; C07D 333/16

(52) U.S. Cl. CPC C07D 409/10; C07D 333/16; C07F 7/1856

(57) ABSTRACT

A process for the preparation of canagliflozin. The process may be effectively implemented on an industrial scale. Several compounds useful as intermediates for the synthesis of canagliflozin (Formula 4, Formula 4a, Formula 4b and Formula 5) are also disclosed. The process involves the reduction of the compound of formula 3 in the presence of a metal hydride and an organic solvent to obtain the compound of formula 4, converting this into a compound of formula 5 which in turn is converted into canagliflozin.

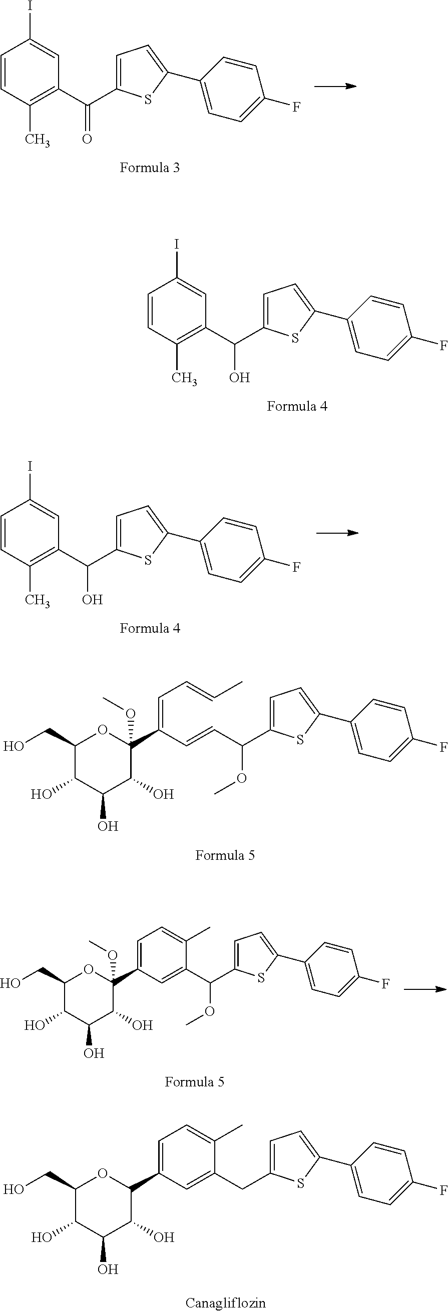

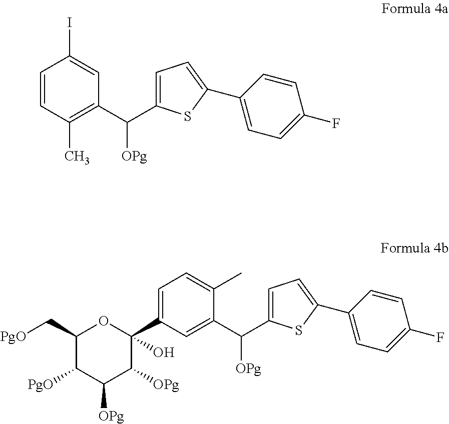

### CROSS-REFERENCE TO RELATED APPLICATIONS

This application, in its entirety, claims the benefit of earlier Indian provisional patent application number 4366/CHE/2014 filed on Sep. 5, 2014.

### FIELD OF THE INVENTION

The present disclosure relates to the field of pharmaceutical sciences and more specifically to a process for the preparation of canagliflozin.

### BACKGROUND OF THE INVENTION

Canagliflozin is an inhibitor of the sodium/glucose transporter 2 (SGLT2) and is indicated as an adjunct to diet and exercise to improve glycemic control in adults with type 2 diabetes mellitus.

IVOKANA® tablets contain canagliflozin. Canagliflozin is chemically known as (2S, 3R 4R, 5S, 6R)-2-{3-[5-[4-fluoro-phenyl)-thiophen-2-ylmethyl]-4-methyl-phenyl}-6-hydroxymethyl-tetrahydro-pyran-3,4,5-triol and has the following chemical structure:

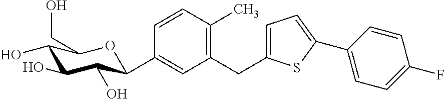

U.S. Pat. No. 7,943,788, which is hereby incorporated by reference, discloses canagliflozin as well as a process for the preparation of canagliflozin.

U.S. Patent Application Publication No. 20090233874, which is hereby incorporated by reference, discloses a crystalline form of canagliflozin and a process for its preparation.

U.S. Pat. No.7,943,582, which is hereby incorporated by reference, discloses a crystalline hemihydrate and amorphous forms of canagliflozin as well as process for its preparation.

PCT Application PCT/CN2010/080357 (WO201179772), which is hereby incorporated by reference, also discloses a process for the preparation of canagliflozin.

The present disclosure provides a novel process for the preparation of canagliflozin which is viable on an industrial scale.

### SUMMARY OF THE INVENTION

One aspect of the present invention provides a process for the preparation of canagliflozin, which may be carried out as shown below in Scheme I:

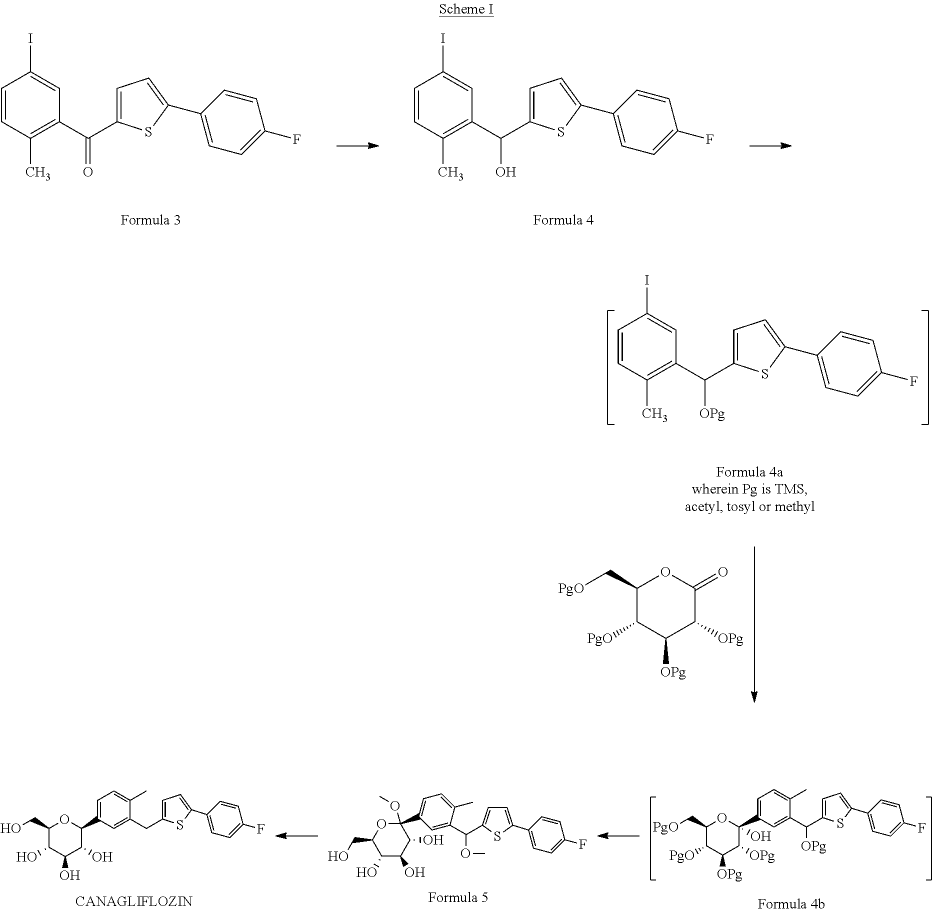

As shown above and throughout the present application, “Pg” is a protecting group. Suitable protecting groups may be, for example, organosilicon-based (for example, trimethylsilyl (TMS)), acetyl, tosyl, or methyl groups.

Another aspect of the present invention provides formula 4, formula 4a, formula 4b, and formula 5, each of which is shown below. These may formulas may be formed during the synthesis of canagliflozin.

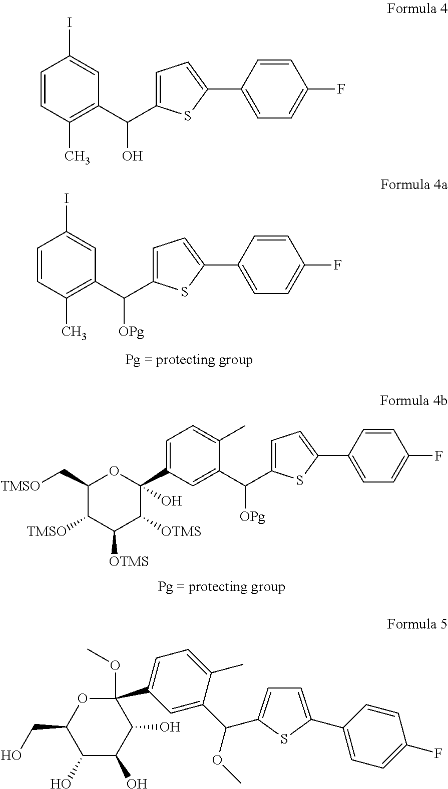

### DETAILED DESCRIPTION OF THE INVENTION

It is to be understood that the figures and descriptions of the present invention have been simplified to illustrate elements that are relevant for a clear understanding of the invention, while eliminating, for purposes of clarity, other elements that may be well known.

The present invention provides a process for the preparation of canagliflozin, which may be carried out as shown below in Scheme I.

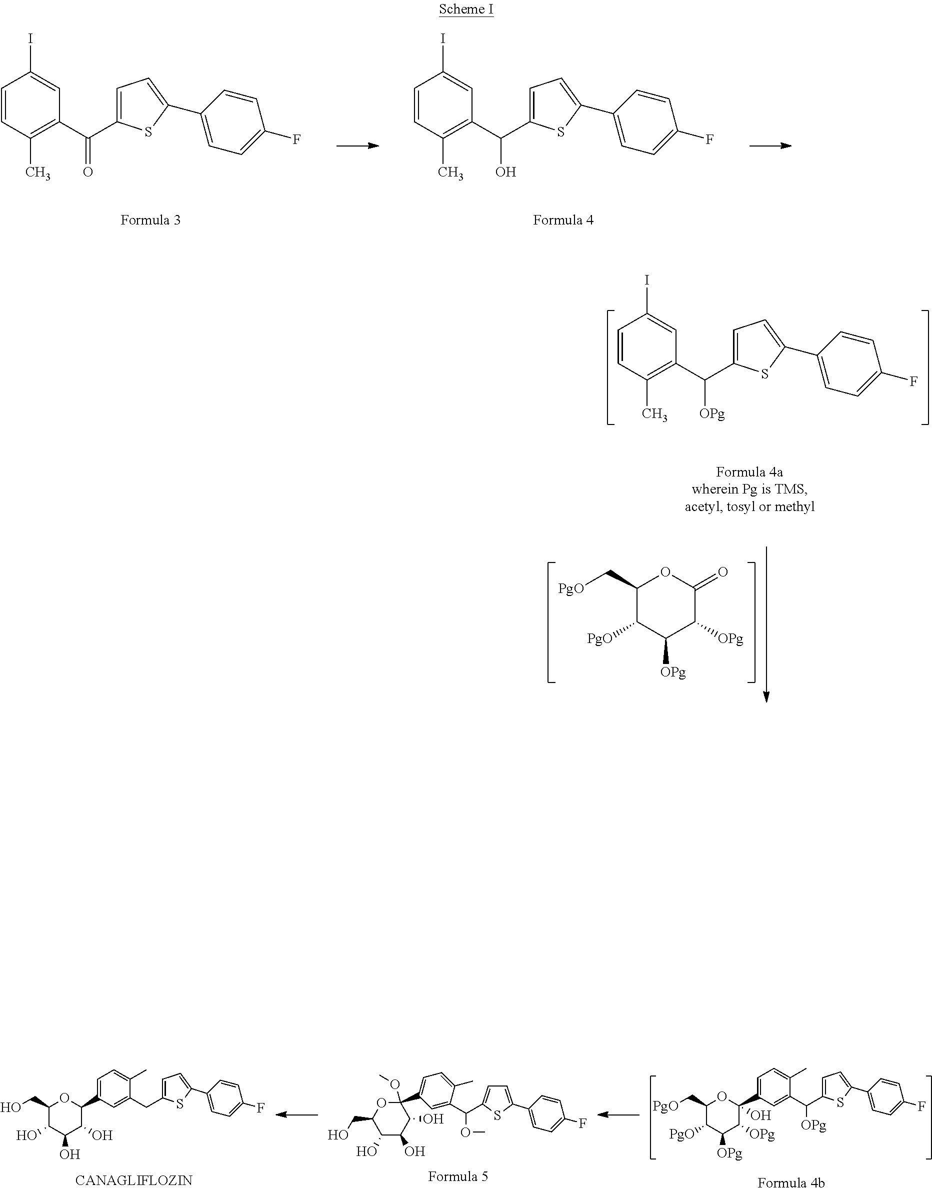

One aspect of the present invention provides a process for the preparation of canagliflozin which may include the following steps:

a) providing formula 3;

b) reducing formula 3 to give formula 4;

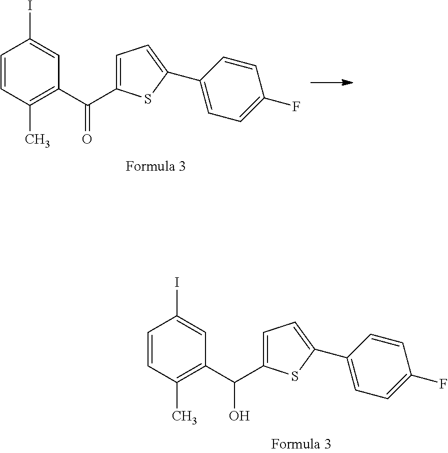 [SMILES: Cc1ccc(I)cc1C(=O)c1ccc(-c2ccc(F)cc2)s1 ; Cc1ccc(I)cc1C(O)c1ccc(-c2ccc(F)cc2)s1]

c) converting formula 4 to formula 5 ; and

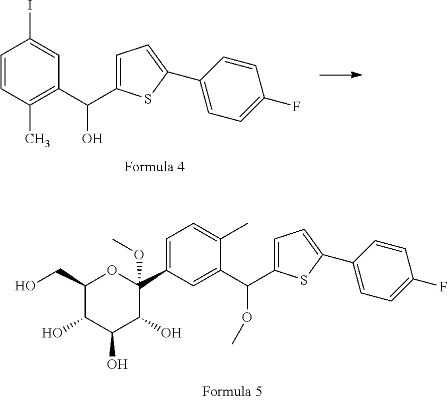

d) converting formula 5 to canagliflozin.

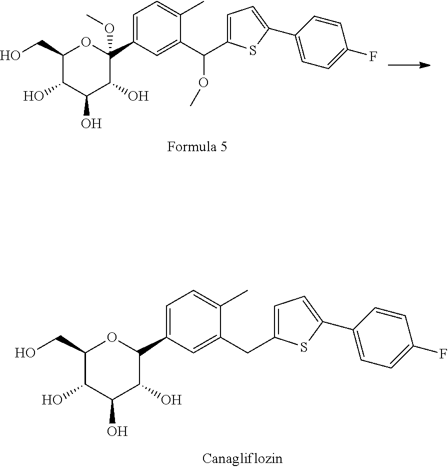

According to the present invention, formula 3 may be reduced to give formula 4. This reaction may be carried out in the presence of a metal hydride and an organic solvent. Within the context of the present invention, examples of suitable metal hydrides include sodium borohydride, diisobutylaluminum hydride, and lithium aluminum hydride. In certain embodiments of the present invention, sodium borohydride was found to be particularly useful. The organic solvent may be, for example, an alcohol. Examples of suitable alcohols include methanol, ethanol, isopropanol, and mixtures thereof.

According to the present invention, formula 4 may then be converted to formula 5. Within the context of the present invention, formula 4a and formula 4b may be formed as intermediates during the conversion of formula 4 to formula 5, as shown in Scheme I above. Suitable protecting groups may be, for example, organosilicon-based (for example, trimethylsilyl (TMS)), acetyl, tosyl, or methyl groups.

Within the context of the present invention, formula 4 may be reacted with a protecting agent to result in formula 4a. Generally, within the context of the present invention, a protecting agent is a reactant that is the source of protecting group residues on the resulting chemical product. Here, the protecting group (“Pg”) is added to formula 4 to generate formula 4a to protect the hydroxyl residue and may thus be characterized as a hydroxyl protecting group. Suitable hydroxyl protecting groups within the context of the present invention include organosilicon-based, tosyl, acetyl, or methyl groups.

Examples of suitable protecting agents include trimethylsilyl chloride, trimethylsilyl iodide, trimethylsilyl bromide, 4-toluenesulfonyl chloride, acetic anhydride, or methyl iodide. One of skill in the art would readily recognize suitable protecting agents and protecting groups as well as conditions for these reactions.

Within the context of the present invention, the conversion of formula 4 to formula 4a may occur in the presence of a base and an organic solvent. The base may be selected so as mediate the addition of the particular protecting group employed. Suitable bases include, for example, N-methylmorpholine, diisopropylethylamine, sodium hydroxide, D-methylaminopyridine (DMAP), or sodium anhydride. The organic solvent may be, for example, tetrahydrofuran, toluene, dichloromethane, dimethyl formamide (DMF), or mixtures thereof. In some embodiments of the present invention, addition of a TMS protecting group in the presence of N-methylmorpholine and tetrahydrofuran was found to be particularly useful for converting formula 4 to formula 4a.

Next, formula 4a may be treated with a protected D-glucolactone, as shown below, to give formula 4b.

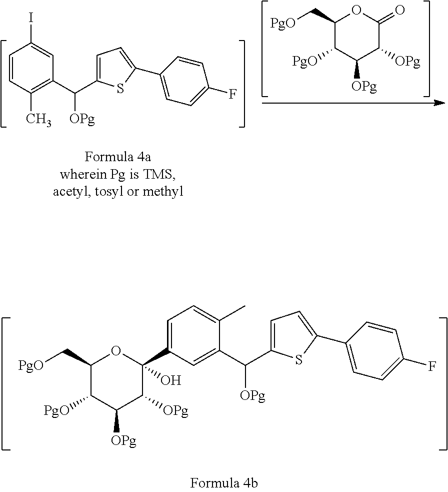

Suitable hydroxyl protecting groups (“Pg” in the scheme above) may be, for example, organosilicon-based (for example, TMS), acetyl, tosyl, or methyl groups. In some embodiments, TMS was found to be a particularly useful protecting group for the D-glucolactone. Within the context of the present invention, this reaction may be performed in the presence of base and an organic solvent. The base may be, for example n-butyllithium, sec-butyllithium, tert-butyllithium, isopropylmagnesium chloride lithium chloride complex, sec-butylmagnesium chloride lithium chloride complex, or (trimethylsilyl)methyllithium. Within the context of the present invention, the organic solvent may be, for example, tetrahydrofuran, toluene, or a mixture thereof. In certain embodiments of the present invention, n-butyllithium was found to be a particularly useful base and tetrahydrofuran was found to be a particularly useful solvent.

According to present invention, Formula 4b may then be converted into formula 5 by reacting formula 4b with a methylating agent in the presence of an organic solvent. The methylating agent may be, for example, methanesulfonic acid. Examples of suitable organic solvents include ethyl acetate, methanol, dichloromethane, toluene, and mixtures thereof. In some embodiments, methanol was found to be a particularly useful solvent.

According to the present invention, formula 5 may then be converted to canagliflozin. This conversion may be carried out in the presence of a reducing agent, a Lewis acid, and an organic solvent. The reducing agent may be, for example, triethylsilane. Examples of suitable Lewis acids include boron trifluoride-ethyl ether complex and aluminum chloride. Suitable organic solvents include, for example, acetone, dichloromethane, ethyl acetate, methyl tert-butyl ether (MTBE), acetonitrile, and mixtures thereof.

Within the context of the present invention, crude canagliflozin may be purified by methods well known in the art, for example, by distillation or by addition of an anti-solvent, to obtain substantially pure canagliflozin. Examples of suitable solvents for distillation include polar solvents such as polar hydrocarbons, ketones, and alcohols. Suitable polar hydrocarbons include dichloromethane, dichloroethane, and mixtures thereof. Suitable ketones include, as examples, acetone, methyl isopropyl ketone, and mixtures thereof. Suitable alcohols include, for example, methanol, ethanol, n-propyl alcohol, isopropanol, n-butanol, and mixtures thereof. Suitable anti-solvents include non-polar hydrocarbons, for example, cyclohexane and n-hexane. One of skill in the art will readily recognize other purification methods that may be used to purify crude canagliflozin.

In the reaction scheme I above, formula 3 is employed as a starting reactant. Within the context of the present invention, formula 3 may be prepared in multiple manners. In one embodiment, formula 3 may be prepared by converting formula 1 to formula 2 and then converting formula 2 to formula 3, as shown below in Scheme 2.

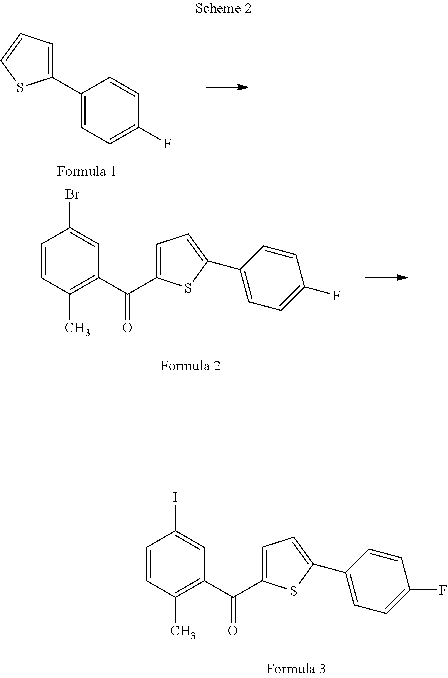 [SMILES: Fc1ccc(-c2cccs2)cc1 ; Cc1ccc(Br)cc1C(=O)c1ccc(-c2ccc(F)cc2)s1 ; Cc1ccc(I)cc1C(=O)c1ccc(-c2ccc(F)cc2)s1]

The conversion of formula 1 to formula 2 may be achieved by preparing two reaction mixtures and combining to result in formula 2. The first reaction mixture may be prepared by first treating 5-bromo-2-methylbenzoic acid with dimethylformamide and oxalyl chloride in the presence of a solvent. Within the context of the present invention, the halogen group on the 5-bromo-2-methylbenzoic acid may be, for example, bromine, fluorine, or chlorine. The second reaction mixture may be prepared by treating formula 1 with a Lewis acid in the presence of a solvent. Within the context of the present invention, the first and second reaction mixtures may be combined to result in the formation of formula 2. Within the context of the present invention, the solvent used to prepare the first and second reaction mixtures may be, for example, dichloromethane, tetrahydrofuran, dioxane, or mixtures thereof. In certain embodiments, dichloromethane was found to be particularly useful. The Lewis acid may be, for example, aluminum chloride.

According to the present disclosure, formula 2 may then be converted into formula 3. This may be achieved by treating formula 2 with copper iodide and an iodide source in the presence of a solvent. Within the context of the present invention, suitable iodide sources include, for example, sodium iodide. Examples of suitable solvents include toluene, diglyme, N,N-dimethylethane-1,2-diamine, and mixtures thereof.

Within the context of the present invention, formula 3 may alternatively be prepared by converting formula 1 directly to formula 3 by reacting formula 1 with 5-iodo-2-methyl benzoic acid or its acid chloride as shown in Scheme III below.

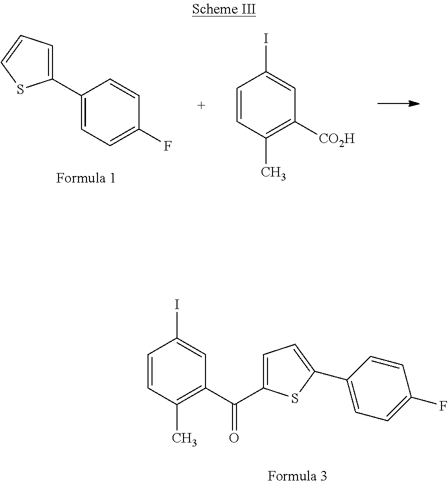 [SMILES: Fc1ccc(-c2cccs2)cc1 ; Cc1ccc(I)cc1C(=O)O ; Cc1ccc(I)cc1C(=O)c1ccc(-c2ccc(F)cc2)s1]

Within the context of the present invention, this reaction may occur in the presence of dimethylformamide, oxalyl chloride, and a solvent. This reaction may produce an intermediate [5-iodo-2-methylbenzol chloride, not shown above] which may then be treated with a Lewis acid in the presence of a solvent. The Lewis acid may be, for example, aluminum chloride. No Suitable solvents include, for example, dichloromethane, tetrahydrofuran, dioxane, and mixtures thereof. In certain embodiments of the present invention, dichloromethane was found to be a particularly useful solvent.

The present invention also provides an alternate process for the preparation of canagliflozin which may be achieved by direct conversion of formula 4b to canagliflozin:

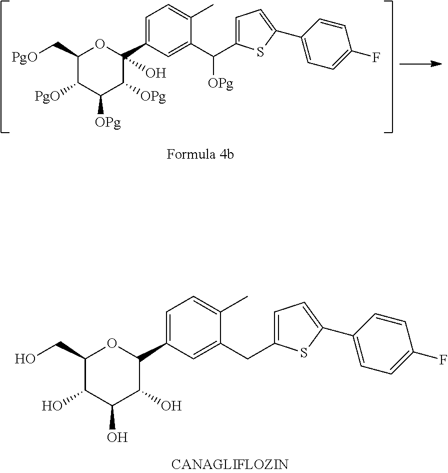

Another aspect of the present invention provides useful intermediates for the production of canagliflozin, including formulas 4, 4a, 4b, and 5. Formula 4 is shown below:

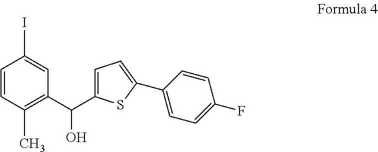 [SMILES: Cc1ccc(I)cc1C(O)c1ccc(-c2ccc(F)cc2)s1]

Another aspect of the present invention provides formula 4a, shown below:

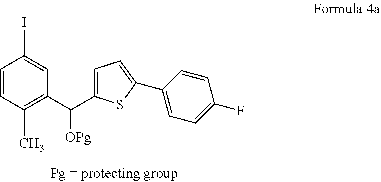

Another aspect of the present invention provides formula 4b, shown below:

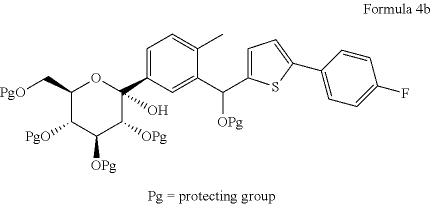

Another aspect of the present invention provides formula 5, shown below:

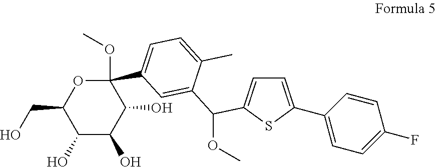 [SMILES: COC(c1ccc(-c2ccc(F)cc2)s1)c1cc([C@]2(OC)O[C@H](CO)[C@@H](O)[C@H](O)[C@H]2O)ccc1C]

As shown in these formulae, “Pg” is a protecting group. The protecting group of formulas 4a and 4b may be, for example, organosilicon-based (for example TMS), acetyl, tosyl, or methyl groups.

The canagliflozin disclosed herein may be incorporated into oral dosage forms, for example, a tablet. Within the context of the present invention, canagliflozin may be incorporated into dosage forms with a variety of excipients well known in the art. Suitable excipients include, for example, croscarmellose sodium, hydroxypropyl cellulose, lactose anhydrous, magnesium stearate, and microcrystalline cellulose. Coatings of formulations in tablet form may contain iron oxide yellow, macrogol/PEG, polyvinyl alcohol, talc, and titanium dioxide. Within the context of the present invention, dosage forms may have about 100 to about 300 milligrams of canagliflozin.

One of skill in the art will be familiar with a variety of excipients and formulations that may be used to prepare desirable dosage forms with desired release characteristics and pharmacokinetic properties without undue experimentation.

When administered to human and non-human patients, formulations of canagliflozin may be adjusted to compensate for the age, weight, and physical condition of the patient. Canagliflozin may be administered over a wide dosage range from about 100 to 300 milligrams per day. Canagliflozin of the present invention may be administered in combination with, prior to, or after dosing regimens of other anti-diabetic compounds, for example, metformin (GLUCOPHAGE®), sulfonylurea, pioglitazone (ACTOS®), and insulin.

When administered to patients, the canagliflozin of the present invention may be useful for improving glycemic control in adults with type-2 diabetes mellitus.

Certain specific aspects and embodiments of the present application will be explained in greater detail with reference to the following examples, which are provided only for purposes of illustration and should not be construed as limiting the scope of the disclosure in any manner.

### EXAMPLES

### Example 1

### Preparation of Formula 2 from Formula 1 (5-bromo-2-methyl-phenyl)-[5-(4-fluro-phenyl)-thiophen-2-yl] methanone

### Step A:

A 500 ml four-necked round bottom flask was charged with 5-bromo-2-methylbenzoic acid (50 g), dichloromethane (200 ml), and dimethylformamide (0.5 g) at 25-35° C. The reaction mixture was cooled to 0-5° C. Oxalyl chloride (30.7 g) was added at 0-5° C. The reaction mass temperature was raised to 25-35° C. After 5 hours, the solvent was distilled off completely under vacuum keeping the temperature below 35° C. The resulting residue (an acid chloride compound) was dissolved in dichloromethane (200 ml) and set aside under nitrogen atmosphere.

### Step B:

Aluminum chloride [AlCl₃] (33.3 g) and dichloromethane (200 ml) were charged at 25-35° C. in a separate 1 L four-necked round bottom flask. The reaction mass was cooled to −10 to 0° C. 2-(4-fluorophenyl) thiophene (Formula 1, 40.4 g) was added under nitrogen atmosphere at −10 to 0° C. After one hour, the residue dissolved in dichloromethane from Step A was added. After one hour, the temperature was raised to 25-35° C. After three hours, the resulting mixture was cooled to −10° C. and quenched with water (50 ml), followed by 2N HCl (45 ml) and hexane (250 ml). Material formation was observed. The resulting material was stirred for 1-2 hours and filtered to give formula 2.

### Example 2

### Preparation of Formula 3 from Formula 2 ((5-(4-fluoro-phenyl) thiophen-2-yl) (5-iodo-2-methyl-phenyl) methanone

A 1 L four-necked round bottom flask was charged with (5-bromo-2-methyl-phenyl)-[5-(4-fluro-phenyl)-thiophen-2-yl]methanone (50 g), sodium iodide (40 g), and copper iodide (1.58 g). The resulting mixture was evacuated and purged with argon. Toluene (250 ml), diglyme (25 ml), and N,N-dimethyl-ethane-1,2-diamine (1.58 g) were then added. The reaction mixture was heated to 110° C. and maintained for 36 hours. Upon consumption of starting material, the resulting mixture was cooled to 45-55° C. Ethyl acetate (200 ml) and carbon were slowly added and the reaction mixture was maintained for one hour at 45-55° C. The carbon was filtered on a Hyflo bed and washed with ethyl acetate (50 ml). The filtrate was taken and the solvent was distilled off completely under vacuum maintaining the temperature below 50° C. Methanol (200 ml) was added and the mixture heated to 60-65° C. The resulting mixture was then cooled to 25-35° C. After three hours, the mixture was filtered to give formula 3.

### Example 3

### Preparation of Formula 4 from formula 3 (5-(4-fluoro-phenyl)thiophen-2-yl) (5-iodo-2-methyl-phenyl)methanol)

### Step A:

A 1 L four-necked round bottom flask was charged with ((5-(4-fluoro-phenyl) thiophen-2-yl) (5-iodo-2-methyl-phenyl) methanone (50 g) and ethanol (500 ml). The resulting mixture was cooled to 0-5° C. Sodium borohydride (8.3 g) was added slowly at 0-5° C. After one hour, the temperature was raised to 25-35° C. and maintained there for 8 hours. The solvent was distilled off completely under vacuum maintaining the temperature below 50° C. Ethyl acetate (500 ml) was then added. The resulting mixture was set aside.

### Step B:

2N HCl (250 ml) was charged in another 1 L four-necked round bottom flask and cooled to 0-5° C. The mixture from Step A was then added. After one hour, the temperature was raised to 25-35° C. After one hour, the aqueous and organic layers were separated and the organic layer was washed with standard sodium bicarbonate solution (100 ml) and brine (100 ml). The organic layer was dried over sodium sulfate and concentrated under vacuum. Isopropanol (100 ml) was then added. The resulting mixture was cooled to 5-10° C. and maintained at that temperature for three hours and filtered to give formula 4 ((5-(4-fluoro-phenyl)thiophen-2-yl) (5-iodo-2-methyl-phenyl)methanol).

### Example 4

### Preparation of Formula 4b from formula 4a ((2S,3R,4S,5R,6R)-2-(3-((5-(4-fluoro-phenyl)thiophen-2-yl)(trimethylsilyloxy) methyl)-4-methyl-phenyl)-3,4,5-tris(trimethylsilyloxy)-6-((trimethylsilyl-oxy)methyl)tetrahydro-2H-pyran-2-ol)

### Step A: Preparation of Formula 4a

A 500 ml four-necked round bottom flask was charged with (5-(4-fluoro-phenyl)thiophen-2-yl) (5-iodo-2-methyl-phenyl) methanol (formula 4, 20 g), 4-methylmorpholine (14.3 g), and tetrahydrofuran (200 ml). The resulting mixture was cooled to 0-5° C. Trimethylsilyl chloride (8.2 g) was slowly added while maintaining the temperature at or below 10° C. After one hour, the reaction mixture was heated to about 35-40° C. for four hours and stirred for overnight at 25-35° C. under argon atmosphere. The resulting mixture was cooled to 0-5° C. Toluene (300 ml) and water (600 ml) were added. After one hour, the temperature was raised to 25-35° C. After one hour, the organic and aqueous layers were separated and the organic layer was washed with aqueous sodium hydrogen phosphate [NaH₂PO₄] (60 ml), water (60 ml), and brine (60 ml). The organic layer was dried over sodium sulfate and concentrated under vacuum to yield ((5-(4-fluoro-phenyl) thiophen-2-yl) (5-iodo-2-methyl-phenyl)methoxy) trimethylsilane (formula 4a) as a light yellow liquid.

### Step B:

A 500 ml four-necked round bottom flask was charged with gluconolactone (30 g), 4-methylmorpholine (136.2 g), and tetrahydrofuran (300 ml). The resulting mixture was cooled to −10 to −5° C. Trimethylsilyl chloride (116.9 g) was slowly added, maintaining the temperature at or below 10° C. After one hour, the reaction mixture was heated to about 35-40° C. for four hours and stirred overnight at 25-35° C. under argon atmosphere. The resulting mixture was cooled to 0-5° C. Toluene (450 ml) and water (850 ml) were added. After one hour the temperature was raised to 25-35° C. After one hour, the organic and aqueous layers were separated and the organic layer was washed with aqueous NaH₂PO₄ (200 ml), water (150 ml), and brine (150 ml). The organic layer was dried over sodium sulfate then concentrated under vacuum to yield 2,3,4,6-tetra-O-trimethylsilyl-β-D-gluconolactone as a light yellow liquid.

### Step C: Preparation of Formula 4b

A 500 ml four-necked round bottom flask was charged under argon with and tetrahydrofuran (150 ml), formula 4a (formed in Step A, 10 g) and the compound formed in Step B (2,3,4,6-tetra-O-trimethylsilyl-β-D-gluconolactone, 12 g). The resulting mixture was cooled to −80 to −70° C. N-butyllithium (40 ml of 1.6 M in hexane) was added dropwise while maintaining the temperature below −70° C. After 30 minutes, the reaction was quenched with standard sodium bicarbonate (20 ml) and allowed to warm to room temperature. The aqueous and organic layers were separated and the organic layer was dried over sodium sulfate and concentrated under vacuum to yield formula 4b ((2S,3R,4S,5R,6R)-2-(3 -((5-(4-fluoro-phenyl)thiophen-2-yl)(trimethylsilyloxy)methyl)-4-methyl-phenyl)-3,4,5-tris(trimethylsilyloxy)-6-((trimethylsilyl-oxy)methyl)tetrahydro-2H-pyran-2-ol) as a thick oil.

### Example 5

### Preparation of Formula 5 from formula 4b ((2S, 3R, 4S, 5S, 6R)-2-(3-((5-(4-fluoro-phenyl)thiophen-2-yl)(hydroxy)methyl)-4-methyl-phenyl)-6-(hydroxy-methyl)-2-methoxytetrahydro-2H-pyran-3,4,5-triol)

A 500 ml four-necked round bottom flask was charged with (2S,3R,4S,5R,6R)-2-(3 -((5-(4-fluoro-phenyl)thiophen-2-yl)(trimethylsilyloxy)methyl)-4-methyl-phenyl)-3,4,5-tris(trimethyl-silyloxy)-6-((trimethylsilyloxy)methyl)tetrahydro-2H-pyran-2-ol (formula 4b, 10g) and methanol (100 ml) under argon atmosphere. The resulting mixture was cooled to 0-5° C. A mixture of methanesulfonic acid (5 g) and methanol (100 ml) were added. After 30 minutes, the temperature was raised to 25-35° C. After 12 hours at 25-35° C., the solvent was distilled off completely under vacuum maintaining the temperature below 45° C. Ethyl acetate (100 ml) and water were added maintaining the temperature below 35° C. The organic and aqueous layers were separated and the organic layer was washed with standard sodium bicarbonate (20 ml), water (20 ml), and brine (20 ml). The organic layer was dried over sodium sulfate and concentrated under vacuum maintaining the temperature below 45° C. Toluene (20 ml) and n-hexane were added still maintaining the temperature below 45° C. The resulting mixture was cooled to 0-5° C. After 3 hours, the mixture was filtered to give formula 5 ((2S, 3R, 4S, 5S, 6R)-2-(3-((5-(4-fluoro-phenyl)thiophen-2-yl)(hydroxy)methyl)-4-methyl-phenyl)-6-(hydroxy-methyl)-2-methoxytetrahydro-2H-pyran-3,4,5-triol).

### Example-6

### Preparation of Canagliflozin from Formula 5

A 500 ml four-necked round bottom flask was charged with (2S, 3R, 4S, 5S, 6R)-2-(3 -((5-(4-fluoro-phenyl)thiophen-2-yl)(hydroxy)methyl)-4-methyl-phenyl)-6-(hydroxymethyl)-2-methoxytetrahydro-2H-pyran-3,4,5-triol (formula 5, 10 g) and dichloromethane (100 ml). The resulting mixture was cooled to −40 to −30° C. Triethylsilane (20 ml) was slowly added keeping the temperature at −40 to −30° C. After 30 minutes, at same temperature, boron trifluoride-ethyl ether complex (15 ml) was added dropwise and the resulting mixture was allowed to warm to 25-35° C. After two hours, the resulting mixture was cooled to 0-5° C. Water (50 ml) was added, and, after 30 minutes, the temperature was raised to 25-35° C. After 30 minutes, the organic and aqueous layers were separated and the organic layer was washed with aqueous sodium bicarbonate (20 ml), water (20 ml) and brine (20 ml). The organic layer was dried over sodium sulfate and concentrated under vacuum to yield canagliflozin as a foam solid.

### Example 7

### Preparation of Formula 3 (5-iodo-2-methyl-phenyl)-[5-(4-fluro-phenyl)-thiophen-2-yl] methanone directly from formula 1

### Step A:

A 500 ml four-necked round bottom flask was charged with 5-iodo-2-methylbenzoic acid (50 g), dichloromethane (200 ml), and dimethylformamide (0.5 g) at 25-35° C. The reaction mixture was cooled to 0-5° C. Oxalyl chloride (30.7 g) was added at 0-5° C. The reaction mass temperature was raised to 25-35° C. After 5 hours, the solvent was distilled off completely under vacuum keeping the temperature below 35° C. The resulting residue (an acid chloride compound) was dissolved in dichloromethane (200 ml) and set aside under nitrogen atmosphere.

### Step B:

Aluminum chloride [AlCl₃] (33.3 g) and dichloromethane (200 ml) were charged at 25-35° C. in a separate 1 L four-necked round bottom flask. The reaction mass was cooled to −10 to 0° C. 2-(4-fluorophenyl) thiophene (Formula 1, 40.4 g) was added under nitrogen atmosphere at −10 to 0° C. After one hour, the residue dissolved in dichloromethane from Step A was added. After one hour, the temperature was raised to 25-35° C. After three hours, the resulting mixture was cooled to −10° C. and quenched with water (50 ml), followed by 2N HCl (45 ml) and heptanes (250 ml). Material formation was observed. The resulting material was stirred for 1 to 2 hours and filtered to give formula 3 ((5-iodo-2-methyl-phenyl)-[5-(4-fluro-phenyl)-thiophen-2-yl] methanone.)

### Example 8

### Preparation of Formula 4 ((5-(4-fluoro-phenyl) thiophen-2-yl) (5-iodo-2-methyl-phenyl)methanol) from formula 3

A 1 L four-necked round bottom flask was charged with (5-iodo-2-methyl-phenyl)-[5-(4-fluro-phenyl)-thiophen-2-yl] methanone (50 g) and dichloromethane (250 ml). The resulting mixture was stirred to result in a clear solution. Sodium borohydride (3.36 g) was added. The resulting mixture was heated to 36-40° C. Methanol (50 ml) was added while maintaining the temperature at 36-40° C. After 30 minutes, the resulting mixture was cooled to 0-5° C. Sodium bicarbonate solution (100 ml) was then added and organic layer was washed with water and brine (100 ml). The organic layer was dried over sodium sulfate and concentrated under vacuum. The formed residue was isolated in a toluene (75 ml) and heptane(s) (25 ml) mixture to give formula 4 ((5-(4-fluoro-phenyl) thiophen-2-yl) (5-iodo-2-methyl-phenyl) methanol).

### Example 9

### Preparation of Formula 5 ((2S, 3R, 4S, 5S, 6R)-2-(3-((5-(4-fluoro-phenyl)thiophen-2-yl)(hydroxy)methyl)-4-methyl-phenyl)-6-(hydroxy-methyl)-2-methoxytetrahydro-2H-pyran-3,4,5-triol)

Preparation of formula 4a: A 500 ml four-necked round bottom flask was charged with (5-(4-fluoro-phenyl)thiophen-2-yl) (5-iodo-2-methyl-phenyl) methanol (formula 4, 20 g), 4-methylmorpholine (14.3 g), and tetrahydrofuran (200 ml). The resulting mixture was cooled to 0-5° C. Trimethylsilyl chloride (8.2 g) was slowly added while maintaining the temperature at or below 10° C. After one hour, the reaction mixture was heated to about 35-40° C. for four hours and stirred for overnight at 25-35° C. under argon atmosphere. The resulting mixture was cooled to 0-5° C. Toluene (300 ml) and water (600 ml) were added. After one hour, the temperature was raised to 25-35° C. After one hour, the organic and aqueous layers were separated and the organic layer was washed with aqueous sodium hydrogen phosphate [NaH₂PO₄] (60 ml), water (60 ml), and brine (60 ml). The organic layer was dried over sodium sulfate and concentrated under vacuum to yield ((5-(4-fluoro-phenyl)thiophen-2-yl) (5-iodo-2-methyl-phenyl)methoxy) trimethylsilane (formula 4a) as a light yellow liquid.

Preparation of 2,3,4,6-tetra-O-trimethylsilyl-β-D-gluconolactone: A 500 ml four-necked round bottom flask was charged with gluconolactone (30 g), 4-methylmorpholine (136.2 g), and tetrahydrofuran (300 ml). The resulting mixture was cooled to −10 to −5° C. Trimethylsilyl chloride (116.9 g) was slowly added maintaining the temperature at or below 10° C. After one hour, the reaction mixture was heated to about 35-40° C. for four hours and stirred overnight at 25-35° C. under argon atmosphere. The resulting mixture was cooled to 0-5° C. Toluene (450 ml) and water (850 ml) were added. After one hour the temperature was raised to 25-35° C. After one hour, the organic and aqueous layers were separated and the organic layer was washed with aqueous sodium hydrogen phosphate (NaH₂PO₄) (200 ml), water (150 ml), and brine (150 ml). The organic layer was dried over sodium sulfate and concentrated under vacuum to yield 2,3,4,6-tetra-O-trimethylsilyl-β-D-gluconolactone as a light yellow liquid.

Preparation of formula 5: A 500 ml four-necked round bottom flask was charged under argon with formula 4a (10 g), 2,3,4,6-tetra-O-trimethylsilyl-β-D-gluconolactone (12 g), and tetrahydrofuran (150 ml). The resulting mixture was cooled to −80 to −70° C. N-butyllithium (40 ml of 1.6 M in hexane) was added dropwise while maintaining the temperature at −70 to −80° C. After 5-10 minutes, a mixture of methanesulfonic acid (5 g) and methanol (100 ml) was added at −70 to −80° C. After 2-4 hours, the resulting mixture was cooled to 10° C. Sodium bicarbonate solution (40 ml) was added, followed by water (50 ml). Sodium hydroxide solution (20 ml) was added to the resulting mixture at 10° C. The salts that were formed were filtered and washed with ethyl acetate. Acetic acid (6 ml) was added, followed by ethyl acetate (150 ml). The aqueous and organic layers were separated and the organic layer was concentrated under vacuum. Dichloromethane (100 ml) was added to the residue and the solution was washed with water (30 ml). The organic layer was dried over sodium sulfate and concentrated under vacuum. The material was isolated with a mixture of toluene (15 ml) and heptane(s) (150 ml) to yield formula 5 ((2S, 3R, 4S, 5S, 6R)-2-(3-((5-(4-fluoro-phenyl)thiophen-2-yl)(hydroxy)methyl)-4-methyl-phenyl)-6-(hydroxy-methyl)-2-methoxytetrahydro-2H-pyran-3,4,5-triol).

### Example 10

### Preparation of Crude Canagliflozin

### Step A:

A 500 ml four-necked round bottom flask was charged with formula 5 (methyl 1-C-(3-{(5-(4-fluorophenyl)thiophen-2-yl](methoxy)methyl}-4-methylphenyl)-D-glucopyranoside, 10 g) and dichloromethane (50 ml) under argon atmosphere. Acetonitrile (40 ml) was added and the mixture was stirred to get clear solution.

### Step B:

Aluminum chloride (12 g) was charged into another round bottom flask under argon atmosphere. Dichloromethane (30 ml) was then added. The resulting mixture was cooled to −3 to −7° C. and acetonitrile (60 ml) was added at −3 to −7° C. followed by the addition of triethylsilane (12 g). The reaction conditions were maintained for 30 minutes at —3 to —7° C. The reaction mass from Step A was added at −3 to −7° C. and maintained for 2.5 to 3.5 hours at −3 to −7° C. The resulting mixture was quenched with water (70 ml). The organic layer was then dried over sodium sulfate and concentrated under vacuum. Material was isolated in a mixture of toluene (15 ml) and heptanes (150 ml). Acetonitrile (24 ml) and water (0.7 ml) was added to the dried material. The temperature of the resulting mixture was raised to 34-38° C. After 30 minutes, heptane (16 ml) was added at 34-3° C. After 2-4 hours, the resulting mixture was cooled to 25-30° C. and maintained for 14-16 hours at 25-30° C. The material was filtered and dried under vacuum to yield crude canagliflozin.

### Example 11

### Preparation of Substantially Pure Canagliflozin

### Step A:

Canagliflozin (10 g) and dichloromethane (50 ml) were added to a 500 ml four-necked round bottom flask. The resulting mixture was heated to 40° C. and distilled completely under vacuum. Dichloromethane (50 ml) was added to the residue and stirred at 40° C. to result in a clear solution. The mass was distilled completely under vacuum. Dichloromethane (20 ml) was added to the solid and the solution was stirred to result in a clear solution.

### Step B:

Cyclohexane (200 ml) was added to a round bottom flask under nitrogen atmosphere. The reaction mass from Step A was added at 25-30° C. and the mixture was stirred for 60 min at 25-30° C. The material was filtered and dried under vacuum at 40-42° C. for 4 hours at which point the temperature of the material was raised to 88-90° C. where it was maintained for 60 min under vacuum. The reaction mass was then cooled to 35-36° C. under vacuum where it was maintained for 6 hours to yield substantially pure canagliflozin.

We claim:

1. A process for the preparation of canagliflozin, comprising the steps of:
a. reducing formula 3 in the presence of a metal hydride and an organic solvent to obtain formula 4; 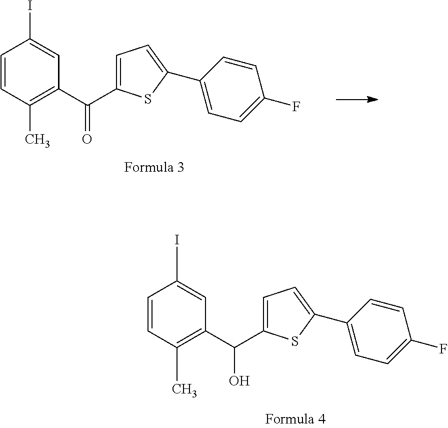 [SMILES: Cc1ccc(I)cc1C(=O)c1ccc(-c2ccc(F)cc2)s1 ; Cc1ccc(I)cc1C(O)c1ccc(-c2ccc(F)cc2)s1]
b. converting formula 4 to formula 5 ; and 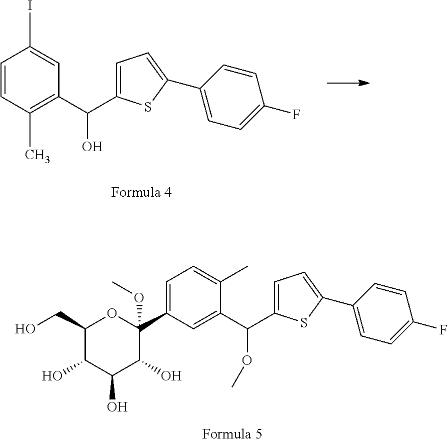
c. converting formula 5 to canagliflozin.

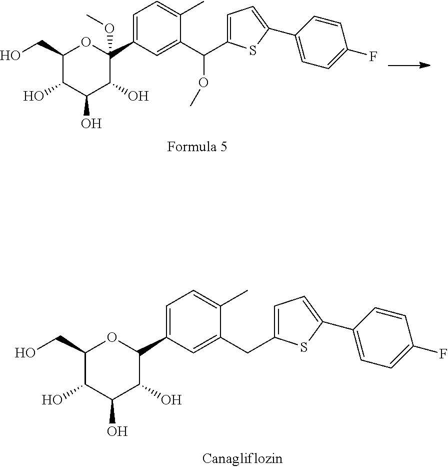

2. The process according to claim 1, wherein the metal hydride is selected from the group consisting of sodium borohydride, diisobutylaluminum hydride, and lithium aluminum hydride.

3. The process according to claim 1, wherein the organic solvent is selected from the group consisting of methanol, ethanol, isopropanol, and mixtures thereof.

4. The process according to claim 1, wherein the converting of formula 5 to canagliflozin is carried out using a reducing agent in the presence of a Lewis acid and an organic solvent.

5. The process according to claim 4, wherein the reducing agent is triethylsilane.

6. The process according to claim 4, wherein the Lewis acid is aluminum chloride or boron trifluoride-ethyl ether complex.

7. The process according to claim 4, wherein the organic solvent is selected from the group consisting of acetone, dichloromethane, ethyl acetate, methyl tert-butyl ether, acetonitrile, and mixtures thereof.

8. The process according to claim 1, further comprising purifying the canagliflozin after converting formula 5 to canagliflozin.

9. The process according to claim 8, wherein the purifying is carried out by distillation or by adding an anti-solvent.

10. The process according to claim 9, wherein the distillation is carried out by adding a polar solvent.

11. The process according to claim 10, wherein the polar solvent is a ketone, an alcohol, or a polar hydrocarbon.

12. The process according to claim 11, wherein the ketone is selected from the group consisting of acetone, methyl isopropyl ketone, and mixtures thereof.

13. The process according to claim 11, wherein the alcohol is selected from the group consisting of methanol, ethanol, n-propanol, isopropanol, n-butanol, and mixtures thereof.

14. The process according to claim 11, wherein the polar hydrocarbon is selected from the group consisting of dichloromethane, dichloroethane, and mixtures thereof.

15. The process according to claim 10, wherein the anti-solvent is a non-polar hydrocarbon.

16. The process according to claim 1, wherein the converting of formula 4 to formula 5 is carried out by the following steps:
a. converting formula 4 to formula 4a;
b. converting formula 4a to formula 4b; and
c. converting formula 4b to formula 5, 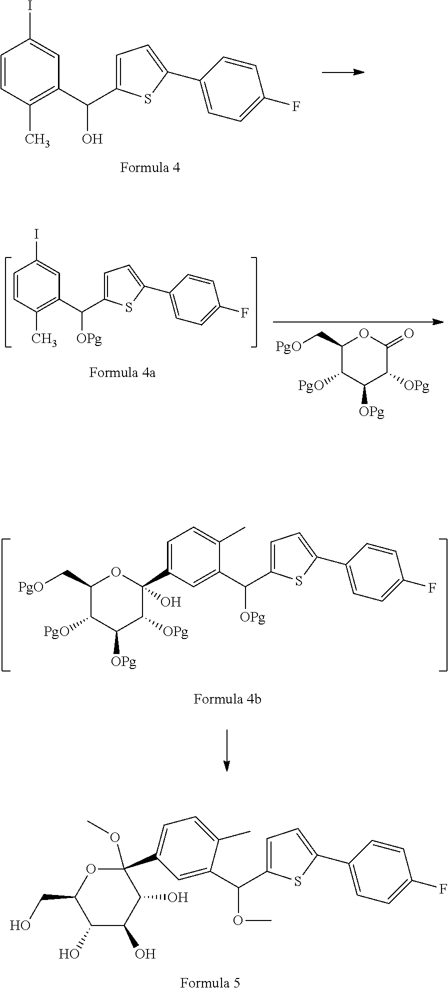
wherein “Pg” is a protecting group.

17. The process according to claim 16, wherein the protecting group is an organosilicon-based group, a tosyl group, an acetyl group, or a methyl group.

18. The process according to claim 16, wherein the converting of formula 4 to formula 4a is done in presence of a protecting agent containing an organosilicon protecting group.

19. The process according to claim 18, wherein the protecting agent is trimethylsilyl chloride.

20. The process according to claim 16, wherein the converting of formula 4 to formula 4a is carried out in the presence of a base.

21. The process according to claim 20, wherein the base is N-methylmorpholine, diisopropylethylamine, sodium hydroxide, D-methylaminopyridine (DMAP), or sodium anhydride

22. The process according to claim 16, wherein the converting of formula 4 to formula 4a is carried out in the presence of an organic solvent.

23. The process according to claim 22, wherein the organic solvent is selected from the group consisting of tetrahydrofuran, toluene, dichloromethane, dimethyl formamide (DMF), and mixtures thereof.

24. The process according to claim 16, wherein the converting of formula 4a to formula 4b is carried out by treating formula 4a with 2,3,4,6-tetra-O-trimethylsilyl-D-gluconolactone in the presence of a base and an organic solvent.

25. The process according to claim 24, wherein the base is selected from the group consisting of n-butyllithium, sec-butyllithium, tert-butyllithium, isopropylmagnesium chloride lithium chloride complex, sec-butylmagnesium chloride lithium chloride complex, and (trimethylsilyl)metyllithium.

26. The process according to claim 24, wherein the organic solvent is selected from the group consisting of tetrahydrofuran, toluene, and mixtures thereof.

27. The process according to claim 16, wherein the conversion of formula 4b to formula 5 is carried out using a methylating agent in the presence of an organic solvent.

28. The process according to claim 27, wherein the methylating agent is methanesulfonic acid.

29. The process according to claim 27, wherein the organic solvent is selected from the group consisting of ethyl acetate, methanol, dichloromethane, toluene, and mixtures thereof.

30. The process according to claim 16, wherein the converting of formula 4a to formula 4b, and the converting of formula 4b to formula 5 are performed in a single reaction mixture without isolation of formula 4b.

31. A compound of formula 4:

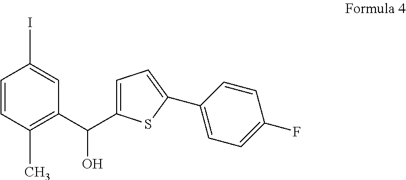 [SMILES: Cc1ccc(I)cc1C(O)c1ccc(-c2ccc(F)cc2)s1]

32. A compound of formula 4a: 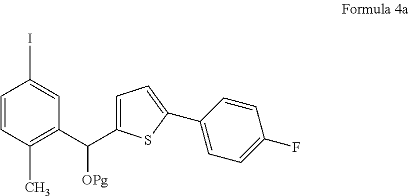
wherein “Pg” is organosilicon-based, acetyl, tosyl, or methyl group.

33. A compound of formula 4b: 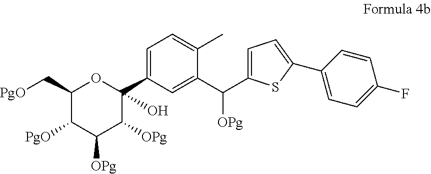
wherein “Pg” is organosilicon-based, acetyl, tosyl, or methyl group.

34. A compound of formula 5: 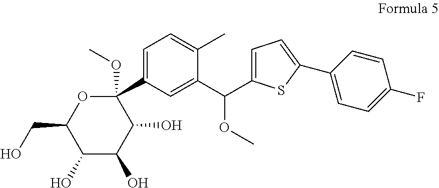 [SMILES: COC(c1ccc(-c2ccc(F)cc2)s1)c1cc([C@]2(OC)O[C@H](CO)[C@@H](O)[C@H](O)[C@H]2O)ccc1C]
